(19) United States
(12) Patent Application Publication
(10) Pub. No.: US 2006/0030937 A1
(43) Pub. Date: Feb. 9, 2006

(54) Carrier and kit for intraluminal delivery of active principles or agents

(75) Inventors: Franco Vallana, Torino (IT); Maria Curcio, Saluggia(Vercelli) (IT); Maria Cristina Cassullo, Santhia(Vercelli) (IT); Andrea Grignani, Chieri(Torino) (IT); Andrea Bottelli, Genova Sestri Ponente (IT)

Correspondence Address: POPOVICH, WILES & O'CONNELL, PA;650 THIRD AVENUE SOUTH, SUITE 600, MINNEAPOLIS, MN, 55402, US

(73) Assignee: Sorin Biomedica Cardio S.p.A.

(21) Appl. No.: 11/249,970

(22) Filed: Oct. 13, 2005

Related U.S. Application Data
(63) Continuation of application No. 10/279,739, filed on Oct. 24, 2002.

(30) Foreign Application Priority Data
Nov. 13, 2001 (EP) EP 01830699.3

(51) Int. Cl. A61F 2/06

(52) U.S. Cl. 623/1.42; 623/1.15

(57) ABSTRACT

A carrier for delivering at least one active principle at an intraluminal site. The carrier includes a carrier body, such as a stent. The carrier body is provided with one or more reservoirs. The reservoirs contain nanoparticles which convey at least one active principle. The nanoparticles also comprise a substance having characteristics of preferential affinity attraction to a desired region at the intraluminal site. The nanoparticles can migrate toward the preferred region.

### FIELD OF THE INVENTION

The present invention relates to intraluminal delivery of active principles or agents. In particular, this invention relates to active agents delivered by stents.

### BACKGROUND OF THE INVENTION

Extensive literature has been devoted to stents. Various stents are described in commonly assigned EP 0 806 190, EP 0 850 604, EP 0 857 470, EP 0 875 215, EP 0 895 759, EP 0 895 760, EP 1 080 738, EP 1 088 528, and EP 1 103 234.

Much current work is directed to developing solutions that enable active or activatable agents of various kinds to be transported on a stent (or on a carrier of a different nature). When stents are used, the agents may be, for example, pharmacological agents, radioactive agents, etc., designed, for instance, to perform an antagonistic function in regard to restenosis. Solutions of the above kind are described, for example, within the above-cited documents, in EP 0 850 604, EP 1 080 738, and EP 1 103 234.

EP 0 850 604 describes the possibility of providing, on the surface of a stent, and in particular on its outer surface, a sculpturing having the function of increasing the surface area of the stent in such a way as to create undercuts and/or, in general, a surface roughness in order to facilitate application of coatings of active or activatable agents. The sculpturing, consisting, for example, of microspheres, may also favor adhesion of the stent to the wall of the vessel being treated.

Again the document EP 0 850 604 envisions the possibility of bestowing on the sculpture in question the aspect of grooves, channels, hollow parts or recesses designed to receive active principles or agents (the latter two terms being used as completely equivalent to one another in the context of the present description).

A solution of the above type is addressed in WO-A-98 23228, EP 0 950 386, and again in commonly assigned, co-pending U.S. application Ser. No. 10/198,054, filed Jul. 18, 2002 (and corresponding to the European patent application 01830489.9), this U.S. application hereby incorporated herein by reference. The solution described in the latter patent application envisions that in the elements of the reticular structure of the stent there are provided recesses that are designed to perform the function of actual reservoirs for receiving agents for treatment of the site of implantation of the stent. Where present, the recesses confer on the respective element a hollowed sectional profile, of which the recesses occupy a substantial portion. The geometry of said recesses is chosen in such a way as to leave unimpaired the characteristics of bending strength of the respective element.

The above solution enables the amount of agent associated with the stent to be sufficient, even when the aim is to obtain a release, and hence an action, that is prolonged in time. To the above there is added the consideration that, in applications of vascular angioplasty, the surfaces of the stent, and above all the inner surface, are subjected to an action of flushing by the blood flow.

Furthermore, the above solution enables the active or activatable agent to be made available and released prevalently, if not exclusively, on the outer surface of the stent, and not, instead, on its inner surface. This is true above all in the case where the agent applied on the stent is designed to perform an antagonistic function in regard to restenosis. The corresponding mechanism of action, which is aimed at acting on the outer surface of the stent facing the wall of the vessel that is undergoing treatment, may in fact have unfavorable effects in areas corresponding to the inner surface; for example, phenomena of neointimal formation on the inner surface of the stent, which are considered to be undoubtedly beneficial in the phases subsequent to the implantation phase, may prove hindered.

This solution thus makes it possible to have available stents that are able to take on the configuration of actual carriers of active or activatable agents, possibly different from one another, which are made available in sufficient quantities to achieve a beneficial effect that may also be prolonged over time, together with the further possibility of making available agents that are even different from one another and are selectively located in different positions along the development of the stent, in such a way as to enable selective variation of the dosages in a localized way, for instance achieving dosages that are differentiated in the various regions of the stent.

The solutions described above hence primarily meets requirements linked to the mechanism of release of the active agent. This applies in particular as regards i) the amount of agent that can be released; ii) the position in which the agent (or the various agents) arranged on the stent is (are) released; and, although to a lesser extent, iii) the time law of delivery/release of the active agent.

Another one of the documents referred to in the introductory part of the present description, namely EP 1 080 738, envisions associating, to the structure of an angioplasty stent, fibres constituting carriers for cores of restenosis-antagonistic agents. In a preferred way, the aforesaid cores are at least in part incorporated in nanoparticles, which are associated to the aforesaid fibres and are provided with an envelope made of bio-erodible material.

The term “nanoparticles” refers in general to corpuscles having a spherical or substantially spherical shape and diameters up to hundreds of nanometers. The nanoparticles in question may present an altogether homogeneous structure, i.e., a so-called “monolithic” structure, formed as a substantially homogeneous dispersion of a particulate substance in a mass having the function of a matrix, or as a core surrounded by an outer envelope. The core and the envelope may have a non-unitary structure, namely, a multiple structure (for example, with the presence of a number of cores or subcores) and/or a stratified structure, even with different formulations from one element to another.

For a more general illustration of the characteristics of the aforesaid nanoparticles, useful reference may be made to the works listed below.
Arshady R; Microspheres and microcapsules: a survey of manufacturing techniques. 1: Suspension and crosslinking. Polym. Eng. Sci. 1989, 30(15): 1746-1758.
Arshady R; Microspheres and microcapsules: a survey of manufacturing techniques. 3: Solvent evaporation. Polym. Eng. Sci. 1989, 30(15): 915-924.
Ruxandra G. et al.; Biodegradable long-circulating polymeric nanoparticles. Science 1994, 263: 1600-1603.
Kreuter J; Evaluation of nanoparticles as drug-delivery systems. I-Preparation method. Pharm. Acta Helv. 1983, 58(7): 196-209.
Narayani R. et al.; Controlled release of anticancer drug methotrexate from biodegradable gelatin microspheres. J. Microencapsulation. 1994, 11(1): 69-77.
Guzman L A. et al.; Local intraluminal infusion of biodegradable polymeric nanoparticles. Circulation 1996, 94: 1441-1448.
Jeyanthi R. et al.; Preparation of gelatin microspheres of bleomycin. International Journal of Pharmaceutics. 1987, 35: 177-179.
Pellizzaro C. et al.; Cholesteryl Butyrate in solid lipid nanospheres as an alternative approach for butyric acid delivery. Anticancer Research. 1999, 19: 3921-3926.
Cavalli R. et al.; Preparation and characterization of solid lipid nanospheres containing paclitaxel. European Journal of Pharmaceutical Sciences. 2000, 10: 305-309.

In particular, in EP 1 080 738 the use is envisioned of nanoparticles of the type comprising at least one core surrounded by an envelope which possibly has a stratified structure. The core comprises an agent that is able to perform an antagonistic function in regard to restenosis as a result of an action of localized release and/or penetration into the wall of the vessel that has undergone stent implantation. The core (or cores) in question may consist, for example, of a drug or a complex of drugs which are provided with an anti-inflammatory action, an anti-mitotic action and/or an action that promotes processes of repair of the wall of the vessel and which are able to mitigate or prevent the reactions that lie at the basis of the restenosis process.

The outer envelope of the nanoparticles consists, instead, of any substance that may be defined as “bio-erodible”, i.e., able to be worn away and/or to assume or present a porous morphology, or in any case a morphology such as to enable diffusion outwards of the substance or substances included in the core. The characteristics of bio-erodibility are typically accompanied by characteristics of biocompatibility and biodegradability.

The substances that can be used for making the envelopes of the nanoparticles according to the aforesaid prior document are, for example, polyethylene glycol (PEG) and polylactic-polyglycolic acid (PLGA). The solution proposed in EP 1 080 738 thus makes it possible to configure the stent as a carrier which, once it is placed in an intraluminal position, is able to perform the function of a true release system, for controlled delivery of restenosis-antagonistic agents. This applies above all as regards the possibility of a precise control of the release kinetics, with the added possibility of selectively controlling release of different agents over time.

Also the solution proposed in EP 1 080 738 thus mainly acts on the mechanism of release of the active agents that can be associated to the stent or to any other type of carrier that can be placed in an intraluminal position.

### SUMMARY OF THE INVENTION

The present invention is directed to solving a problem which is, to a certain extent, complementary to that described in the prior art, namely, that of controlling the kinetics of release of the active agents also as regards control of the interaction with the intraluminal site in which the carrier is placed, namely, in the case of stents (an example to which reference will continue to be made in the remaining part of the present description), the part of the vessel in which the stent is implanted and the surrounding regions.

According to the present invention, the above problem is solved by means of a carrier for intraluminal delivery of active agents which has the characteristics described below. The invention also relates to the corresponding kit, comprising a carrier of the above-specified type combined with an inserter means for placing the carrier in an intraluminal site. Preferably, the inserter means is a catheter, and, even more preferably, a balloon catheter.

Substantially, the solution according to the invention is largely based upon the composition of the nanoparticles, and preferably upon the composition of the envelope and/or upon its thickness, both with a view to obtaining a more or less fast release of the active principle contained therein and with a view to enabling the nanoparticles and agents contained in the envelopes to be selectively “guided” towards given areas or regions, more especially towards particular types of tissue of the environment surrounding the carrier, thus achieving a sort of selective attraction of the active principles by the areas (tissues, organs, etc.) that function as targets. In other words, the nanoparticles are provided with a sort of force of attraction that guides them in the direction of the target. The invention thus creates a release system that has a very high degree of efficiency, with the consequent possibility of reducing the absolute amount of active agent or principle that is to be administered.

Although the present invention has been developed with particular attention paid to its possible application to stents, it will be evident to a person of skill in the art that its scope is altogether general, and consequently the invention may be applied to any type of carrier that is designed to be placed in an intraluminal position (i.e., inside any vessel of the human body), for example by means of catheterization.

In one aspect, this invention is a carrier for delivering at least one active principle at an intraluminal site, the intraluminal site having at least a first region and a second region, the carrier comprising a carrier body sized to be conveyed to the intraluminal site, the carrier body having at least one reservoir; and a plurality of nanoparticles contained within the at least one reservoir, each nanoparticle including an outer envelope and containing the active principle, the outer envelope comprising at least a first substance having characteristics of affinity of preferential attraction to the second region as compared to the first region.

In another aspect, this invention is a stent for delivering at least one active principle at an intraluminal site, the intraluminal site having at least a first region and a second region, the stent comprising a body configured to be expandable from a delivery configuration to a deployed configuration, the body being sized to be delivered to the intraluminal site in the delivery configuration, the body having an interior surface and an exterior surface and having at least one reservoir on the exterior surface; and a plurality of nanoparticles contained within the at least one reservoir, each nanoparticle including an outer envelope and containing the active principle, the outer envelope comprising at least a first substance having characteristics of affinity of preferential attraction to the second region as compared to the first region.

In a third aspect, this invention is a method for delivering at least one active principle at an intraluminal site, the intraluminal site having at least a first region and a second region, the method comprising providing a stent having a body configured to be expandable from a delivery configuration to a deployed configuration, the body having an interior surface and an exterior surface and having at least one reservoir on the exterior surface; placing in the at least one reservoir a plurality of nanoparticles, each nanoparticle including an outer envelope and containing the active principle, the outer envelope comprising at least a first substance having characteristics of affinity of preferential attraction to the second region as compared to the first region; delivering the stent in its delivery configuration to the intraluminal site; and expanding the stent to its deployed configuration at the intraluminal site. The stent may be delivered by a catheter.

In a fourth aspect, this invention is a kit for delivering at least one active principle at a treatment site within the lumen of a vessel, the site having at least a first region and a second region, the kit comprising a carrier body sized to be conveyed through the lumen of the vessel to the treatment site, the carrier body having at least one reservoir; a plurality of nanoparticles contained within the at least one reservoir, each nanoparticle including an outer envelope and containing the active principle, the outer envelope comprising at least a first substance having characteristics of affinity of preferential attraction to the second region as compared to the first region; and a delivery device for advancing the carrier body through the lumen to the treatment site. The delivery device may be a catheter, such as a balloon catheter.

In preferred embodiments, each nanoparticle comprises a core which itself comprises the active principle. The outer envelope is permeable to the active principle and may comprise bio-erodible material and may also have a stratified structure. There may be a plurality of reservoirs that can contain at least two different species of nanoparticles.

### BRIEF DESCRIPTION OF THE DRAWINGS

The present invention will now be described, purely by way of non-limiting example, with reference to the attached drawings, in which:

FIG. 1 is a schematic illustration of the characteristics of nanoparticles that can be used in the framework of the invention;

FIG. 2 schematically illustrates the operating principle of the invention applied to an angioplasty stent;

FIGS. 3 to 10 schematically illustrate, in cross-section, different modes of use of the invention, again applied to an angioplasty stent; and

FIG. 11 is a partial planar view of a stent of this invention.

### DETAILED DESCRIPTION OF THE PREFERRED EMBODIMENTS

As previously stated, although the present invention will be described in connection with its application to stents, in particular to angioplasty stents, its range of application is altogether general. The solution according to the invention can be applied to any carrier which can be placed, for example by means of catheterization, in an intraluminal position, i.e., inside a vessel of the human body or of the body of an animal which is to undergo a type of treatment that involves, as a main step or as an accessory step, delivery of an active principle or agent, for instance in the form of a drug.

On the basis of the above introductory remarks it will be understood that the invention can be applied, for example (and without the possibility of the ensuing list being considered in any way limiting), in addition to stents, such as angioplasty stents, to vascular grafts, to the so-called stents/grafts, to catheters for percutaneous coronary balloon angioplasty (PTCA) treatments, catheters for mechanical/electrical ablation of endovascular plaques, catheters or electrodes for the elimination or passivation (again by mechanical, electrical and/or chemical means) of the so-called ectopic foci responsible for fibrillation phenomena, electrodes for electrostimulation/defibrillation, electrodes for endocardial mapping, endoscopes and similar devices.

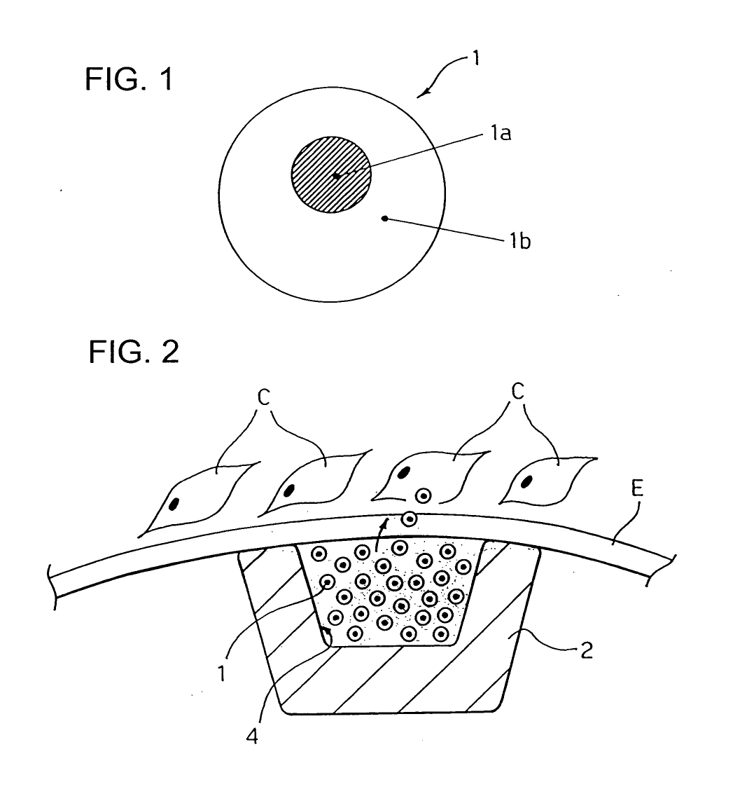

FIG. 1 illustrates the characteristics of a structure 1 of the type currently referred to as “nanoparticle”. By this name is generally meant (see in this connection the references quoted in the introductory part of the present description) corpuscles having a spherical or substantially spherical shape and a diameter on the order of hundreds of nanometers. In one embodiment of the invention herein illustrated, nanoparticles 1 usually comprise core 1a surrounded by outer envelope 1b.

FIG. 1 shows that the core 1a, instead of being in a substantially central position, may be in an eccentric position with respect to the envelope 1b. Again, while FIG. 1 shows a nanoparticle comprising a single core 1a, it is possible to obtain nanoparticles 1 that have a multiple structure (for example, with the presence of a number of cores or subcores). And again, while FIG. 1 shows an envelope 1b with a substantially uniform structure, it is possible to obtain envelopes 1b having a stratified structure.

The core 1a may be made or may comprise any agent (the term being used herein in its widest sense, and hence may comprise any active/activatable principle or any drug) which is able to perform an action, in particular a local action, on the site where the corresponding carrier (illustrated in greater detail hereafter) is placed in an intraluminal position. To clarify the concept (without this being viewed in any way as limiting the scope of the invention), the agent or agents that make up the core or cores 1a of the nanoparticles 1 or that are comprised therein may consist of a drug or a complex of drugs with an anti-inflammatory action, such as the ones listed below.

Corticosteroids:

| | | |
|---|---|---|
| Cortisol | Betamethasone | Fluocinolone |
| Cortisone | Dexamethasone | Fluocinonide |
| Corticosterol | Flunisolide | Fluorometholone |
| Tetrahydrocortisol | Alclomethasone | Fluorandrenolide |
| Prednisone | Amcinonide | Alcinonide |
| Prednisolone | Clobetasol | Medrisone |
| Methylprednisolone | Clocortolone | Momethasone |
| Fluodrocortisone | Desonide | Rofleponide |
| Triamcinolone | Desoxymethasone | |
| Paramethasone | Diflorasone | |

as well as all the corresponding esters, salts and derivatives.

NSAIDs (Non-Steroidal Anti-Inflammatory Drugs):

NSAIDs (non-steroidal anti-inflammatory drugs):

| | |
|---|---|
| Salicylates: | Acetyl salicylic acid |
| | Diflunisal |
| | Salsalate |
| Pyrazolones: | Phenylbutazone |
| | Oxyphenbutazone |
| | Apazone |
| Indomethacin | |
| Sulindac | |
| Mefenamic acid and fenamates | |
| Tolmetin | |
| Derivatives of propionic acid: | Ibuprofen |
| | Naproxen |
| | Phenoprofen |
| | Ketoprofen |
| | Flurbiprofen |
| Pyroxicam and its derivatives | |
| Diclofenac and its derivatives | |
| Etodolac and its derivatives | |

In addition or as an alternative, the active agent or principle may comprise a drug or a complex of drugs with antineoplastic action, such as the ones listed below.

Alkylating Agents:

Alkylating agents:

| | Nitrogen mustards: | Cyclophosphamide |
|---|---|---|
| | | Melfalan |
| | | Chlorambucile |
| | Ethylenimine and methylmelamine | |
| | Alkyl sulphonates | |
| | Nitrosureas: | Carmustine |
| | Triazenes | |
| Antimetabolites: | | |
| Analogs of folic acid: | | Methotrexate |
| Analogs of pyrimidine: | | Fluorouracyl |
| Analogs of purine and derivitives | | Mercaptopurin |
| thereof: | | Thioguadinine |
| Natural products: | | |
| | Alkaloids of Vinca: | Vinblastine |
| | | Vincristine |
| | Epipodophyllotoxins: | Etoposide |
| Antibiotics: | | |
| | | Actinomycin D |
| | | Doxoribicine |
| Various: | | |
| | Complexes of platinum: | Cisplatinum |

Mithoxandrone and its derivatives
Hydroxyurea and its derivatives
Procarbazine and its derivatives
Mitotanes
Aminoglutetimide
Derivatives with napthopyrane structure
Derivatives of butyric acid

| | | |
|---|---|---|
| | Taxanes: | Taxol |
| | | Docetaxel |
| Epotilones | | |

Batimastat and its analogues

In addition or as an alternative, the active agent or principle may comprise a drug or a complex of drugs with an action that promotes processes of repair of the vessel wall, such as endothelial/angiogenic growth factors (VEGF) or antisense oligonucleotides.

In addition or as an alternative, the active agent or principle may comprise a drug or a complex of drugs that is able to mitigate or prevent the reactions lying at the root of the process of restenosis of a vessel that has undergone stent implantation, such as:

| | |
|---|---|
| Rapamycin | Heparin and the like |
| Actinomycin D | Batimastat |
| Paclitaxel | Resten-NG (oligonucleotide) |
| Dexamethasone | |

Other active principles or agents that can be used in the framework of the present invention include, for example:
Antisense oligonucleotides: e.g., antisense c-myb
Prostacyclines and analogues thereof: Ciprostene
Dipyridamole
Calcium channel blockers:
   Arylalkyl amines: Diltiazem, Verapamil, Fendiline, Gallopamil, etc.
   Dihydropyridines: Amlodipine, Nicarpidine, etc.
   piperazines: Cinnarizine, Lidoflazine, etc.
Colchicine
Drugs that act on c-AMP:
   Aminophylline, IBMX (bronchodilators)
   Amrinone (cardiotonic)
   8-Bromo-c-AMP and analogues of c-AMP
Drugs that act on lipid metabolism:
   Statins: simvastatin, fluvastatin, etc.
   Unsaturated ω-3 fatty acids
Somatostatins and analogues thereof. Sandostatin, Angiopeptin, etc.
Cytochalasin
Etretinate and derivatives of retinoic acid

Immunosuppressors:

| | |
|---|---|
| Cyclosporins | Rapamycin |
| Tacrolimus | Leflunomide |
| Mycophenolate | Brequinar |

Anticoagulants: Hirudin, Heparin and derivatives thereof.
Trapidil: vasodilator
Nitrogen monoxide and its generators: Molsidomine
Platelet inhibitors: Ticlopidine, Dipyrimidamole, etc.
Agents that may act on the activity of the cell and on the regulation of the cell matrix:
   proteins (elafin)
   oligonucleotides
   genes
   RNA, DNA and fragments thereof
   RNA, DNA and antisense fragments thereof
   monoclonal antibodies

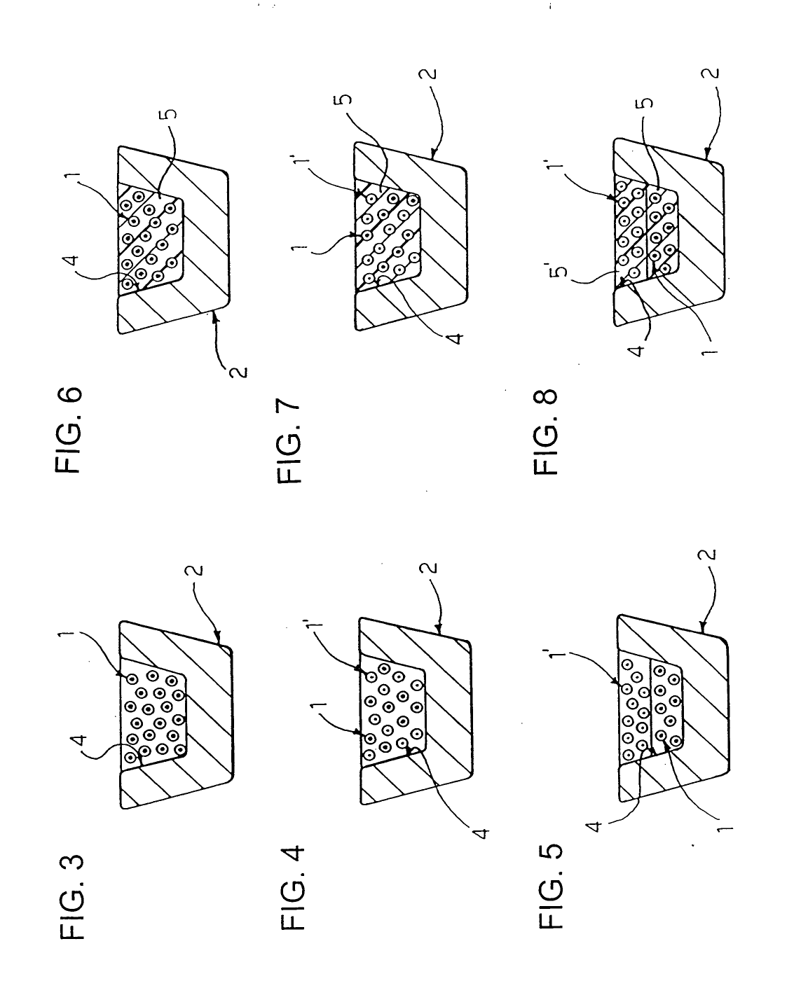

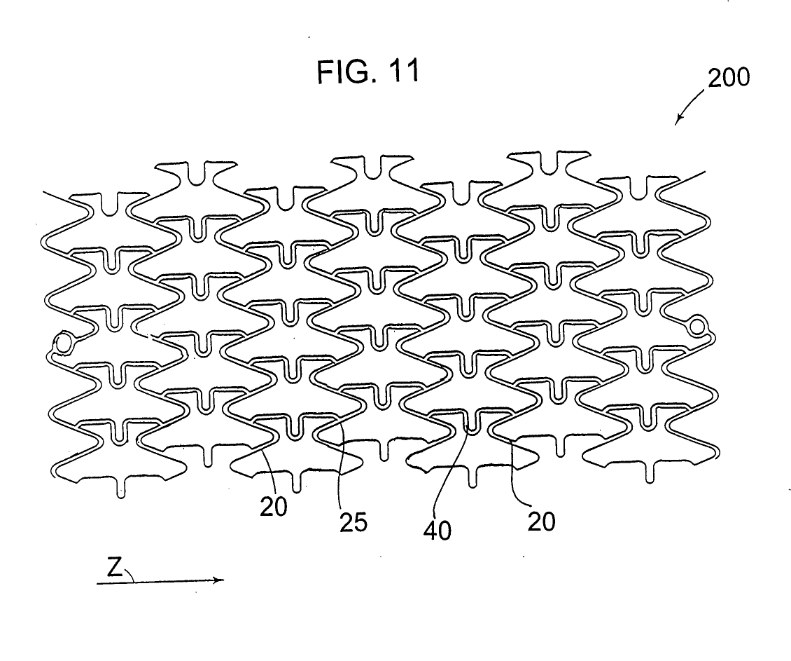

Before passing on to a more detailed illustration of the characteristics of the envelope 1b of the nanoparticles 1, useful reference may be made to the general scheme of FIG. 2. In this figure, the reference number 2 designates one part of the structure of a stent of any known type which is shown in cross-section. The stent comprises a tubular body which is radially expandable and is formed by elements or “struts” that define a reticular structure. The stent may be, for example, of the type illustrated in the document EP 0 875 215 as generally represented in FIG. 11.

FIG. 11 shows a partial planar view of stent 200. When in use, the stent has a roughly cylindrical shape. Stent 200 comprises a plurality of annular elements 20 which have a serpentine pattern. These annular elements are designed to be aligned in sequence along the main axis of the stent designated as the Z axis. Annular elements 20 are connected together by means of longitudinal connection elements 40, generally referred to as “links” or “bridges” and have, in the example of embodiment illustrated in the document EP 0 875 215 and in FIG. 11 a general lambda conformation. Preferably, the aforesaid connection elements 40 are connected to the cylindrical elements of the stent at the zero points (shown at 25) of the respective sinusoidal paths

In any case, the geometrical details of the stent do not constitute a limiting or binding element of the invention; the solution according to the invention can, in fact, be applied to stents of any type, shape or size. Even though the invention has been developed with particular attention paid to its possible use in the sphere of stents obtained starting from a microtube, the solution according to the invention can also be applied to stents obtained, for instance, starting from variously shaped filiform materials (the so-called “wire stents”).

More in general, it is recalled once again that the solution according to the invention can in general be used together with any carrier that is designed to be placed in an intraluminal position.

Referring again to FIG. 2, the elements 2 of the stent, which have in general a filiform or bar-like configuration, are provided, preferably on the surface of the stent facing outwards, with recesses or reservoirs, designated as a whole by 4. Such recesses or reservoirs are similar to those proposed in EP 0 850 604 (FIGS. 6 and 7) and developed in the European patent 01830489.9.

The recesses or reservoirs in question may either basically amount to a single recess which extends, practically without any discontinuities, over the entire development of the stent, or be chiefly, if not exclusively, made in areas corresponding to the rectilinear, or substantially rectilinear, portions of the branches of the stent, thus avoiding in particular both the curved parts (for example, the cusp or loop parts of the elements in question) and the areas in which the connection elements or links are connected to the various annular elements that make up the stent. In particular, formation of the aforesaid recesses or reservoirs may be limited just to the areas of the elements of the stent that will be less subject to stress during operation of the stent.

Again, the recesses or reservoirs 4 may be made in the form of separate wells set at a distance apart from one another and variously distributed over the surface of the stent. The characteristics of implementation of the recesses described above may, of course, also be used in combination with one another. Consequently, it is possible to have, in one and the same stent, both recesses that extend practically without any discontinuities over an entire portion of the stent and recesses consisting of slits or wells.

However made, the recesses in question are such as to constitute hollowed-out formations which can function as reservoirs to enable arrangement of active/activatable agents, possibly of different types, on the stent. For example, in the case where recesses or reservoirs 4 have a general well-like conformation, each of the wells constitutes a recess for receiving within it an active/activatable agent having different characteristics. The foregoing affords the possibility of having available on the stent—at least virtually or in principle—as many different agents as there are recesses.

Additionally, the recesses or reservoirs can be used to accommodate different agents in different areas of the stent. For instance, the recesses located at the ends of the stent can receive anti-inflammatory agents since the end parts of the stent are the ones most exposed to the possible onset of inflammatory phenomena. This means that at least one first agent with anti-inflammatory characteristics is present in a higher concentration at the ends of the stent as compared to the central area of the stent. The possibility may then be envisioned of distributing another agent, such as an anti-mitotic agent, with a level of concentration that is constant throughout the longitudinal development of the stent, with the added possibility of distributing yet another agent, such as a cytotoxic or cytostatic agent, with a maximum level of concentration in the central area of the stent and levels of concentration that progressively decrease towards the ends of the stent.

Irrespective of the modalities of construction of the recesses or reservoirs 4, it may immediately be realized that the presence of the recesses or reservoirs 4, preferably made on the outer surface of the stent, makes available a wide reservoir for gathering active/activatable agents that can be released from the stent towards the adjacent tissue, which, as shown in FIG. 2, is in the form of the endothelium E and of the cells C of the smooth muscle.

Since the recesses or reservoirs 4 are made preferably in the outer surface of the stent, the phenomenon of release takes place preferably in a centrifugal direction, i.e., from the outside of the stent 1 towards the wall of the vessel undergoing treatment. The modalities of construction of the recesses or reservoirs 4 herein illustrated thus make it possible to contain to a very marked extent the phenomena of possible diffusion in a radial direction towards the inside of the stent 1. In this way, it is possible to prevent undesired antagonistic phenomena in regard to the possible neointimal formation.

Again, the fact of having available recesses or reservoirs 4 of large dimensions renders less critical the aspect linked to the physical anchorage of the agent or agents to the surface of the stent. This aspect is particularly important in so far as it makes it possible to apply on the surface of the stent (with the possible exclusion of the surface of the recesses or reservoirs 4, even though this fact is not of particularly determining importance) a layer of biocompatible carbon material (not specifically illustrated in the drawings). This may be, for example, a coating of the type described in the documents U.S. Pat. No. 5,084,151 (Vallana et al.), U.S. Pat. No. 5,133,845 (Vallana et al.), U.S. Pat. No. 5,370,684 (Vallana et al.), U.S. Pat. No. 5,387,247 (Vallana et al.) and U.S. Pat. No. 5,423,886 (Arru et al.). A coating of carbon material of this sort performs an anti-thrombogenic function, favoring endothelialization and, a factor that is deemed of particular importance, acting in the direction of preventing release of metal ions from the stent 1 to the surrounding tissue.

According to another feature of the invention, the desired active agents are transported by means of nanoparticles 1, as described in greater detail below.

In particular, it is envisioned that the material of the envelope 1b should be chosen in such a way as to present specific characteristics of selective affinity in regard to organs (or more in general, tissues or regions) that act as targets, the aim being that the nanoparticles, and hence the active principles carried thereby, should concentrate in a selective, and hence differentiated, way in the target regions. In practice, the nanoparticles 1 behave as if they were provided with a sort of driving force that guides them to the target region.

It is thus possible to give rise to a delivery system, which, precisely on account of its selectivity, presents a very high efficiency, with a consequent reduction in the absolute amount of active principle that is to be administered, and hence to be transported by means of the carrier (e.g., by the stent).

In the present case, the target region or regions comprises different types of tissue according to the illness that is to be treated. For example, when a restenosis-antagonistic function is to be performed, the target region is chiefly represented by the cells C of the smooth muscle that surrounds the endothelium E of the vessel.

Consequently, the solution described has a degree of efficiency—and hence a precision of treatment, also as regards local diffusion of the active agent exclusively towards the organs that are to be treated—which is considerably higher than that of traditional solutions. In the traditional solutions in question, the active principle (for example, rapamycin in the case of a restenosis-antagonistic cytostatic function) is released by diffusion, from polymeric matrices arranged on the stent, throughout the environment (blood, first of all, and then plaque and vessel) that surrounds the stent.

Preferably, the envelope 1b of at least some of the nanoparticles 1 is made of a bio-erodible material and/or a material permeable to the active principle that constitutes the core 1a of the respective nanoparticle. Yet again, the envelope 1b of at least some of the nanoparticles may present a stratified structure. Of course, the representation of FIG. 2, in which nanoparticles 1 may be seen that are arranged in such a way as to constitute a mere filling of the recess 4 is to be held purely an example. In particular, the aim of FIG. 2 is to illustrate the mechanism of action of the nanoparticles; see in particular the nanoparticles illustrated already in the position of migration through the endothelium E and inside one of the cells C.

By way of example, assume that the aim is to transport to the cells C an active principle, e.g., rapamycin, an immunosuppressor, at the same time containing and virtually preventing transport of the agent towards and within the endothelium E. In this case, the active principle is included in the cores 1a of the nanoparticles 1, and in the envelopes 1b of the nanoparticles 1 themselves there are instead provided functional groups of recognition of the muscle cells C, such as peptide sequences or proteins of recognition (antibodies) or fractions/fragments thereof. A specific example in this connection is represented by the sequences of the type arginine-glycine-aspartic acid (RGD).

The above mechanism of selective delivery/diffusion of the active principle to the cells C is therefore linked to the fact that the nanoparticles are provided with envelopes 1b having differentiated characteristics of affinity attraction in regard to the various regions (hence to the various organs) corresponding to the site of implantation of the carrier.

When the carrier is located in the site of implantation, each nanoparticle migrates primarily and selectively towards a region (namely, towards an organ) in regard to which the nanoparticle has greater affinity attraction, thus giving rise to a selective mechanism of delivery of the active principle or active principles carried thereby.

The above characteristic can be exploited for providing, in the recesses or reservoirs 4 of the carrier, both fillings of nanoparticles of a homogeneous type and fillings of nanoparticles comprising nanoparticles of at least one first species and one second species, which are different from one another.

For example, assume that (in addition to selectively delivering rapamycin to the cells C) the aim is to deliver to the endothelium E an agent (for example, VEGF, the endothelial/angiogenic growth factor) aimed at promoting re-growth of the intima of the endothelium E itself, at the same time preventing (or at least containing) delivery/diffusion of the active principle to the cells C.

In this case, in addition to the nanoparticles 1 seen previously, it is possible to envision the presence, in the recess or recesses 4, of a second species of nanoparticles 1, the cores 1a of which transport the agent VEGF, whilst the corresponding envelopes 1b are substantially of a lipidic nature, consisting, for example, of stearic acid. There is thus obtained a preferential, and hence selective, administration of the agent VEGF in the endothelium E (and in particular in the first layers facing the stent), at the same time obtaining preferential and selective delivery of rapamycin to the cells C.

In a preferred embodiment, the carrier has a plurality of reservoirs or recesses. Each reservoir may contain the same kind of nanoparticle, i.e., wherein all the nanoparticles have the same characteristics and comprise the same active principle. The reservoirs may contain different kinds of nanoparticles. Alternatively, more than one kind of nanoparticles may be in one reservoir. The aforesaid mechanisms of differentiation of the species of nanoparticles within the individual recess or in the framework of different recesses can be used in a combined way, in particular in different regions of the stent, if necessary again exploiting other factors, such as the possibility of dispersing the active principles within polymeric matrices, in particular of a bio-erodible type. Such an approach may be particularly useful if nanoparticles having the desired characteristics are used in different locations on the carrier. In this way, active principle can be delivered only to a desired region.

The invention thus allows for considerable flexibility in placing active principles or agents on a carrier. For example, a carrier may have a first reservoir containing only nanoparticles comprising an active agent A, a second reservoir containing nanoparticles of different kinds comprising active agents A and B and a third reservoir which contains only nanoparticles comprising active agent B. By selecting the location on the carrier where the various nanoparticles are contained, it is possible to deliver a desired active agent at a desired location.

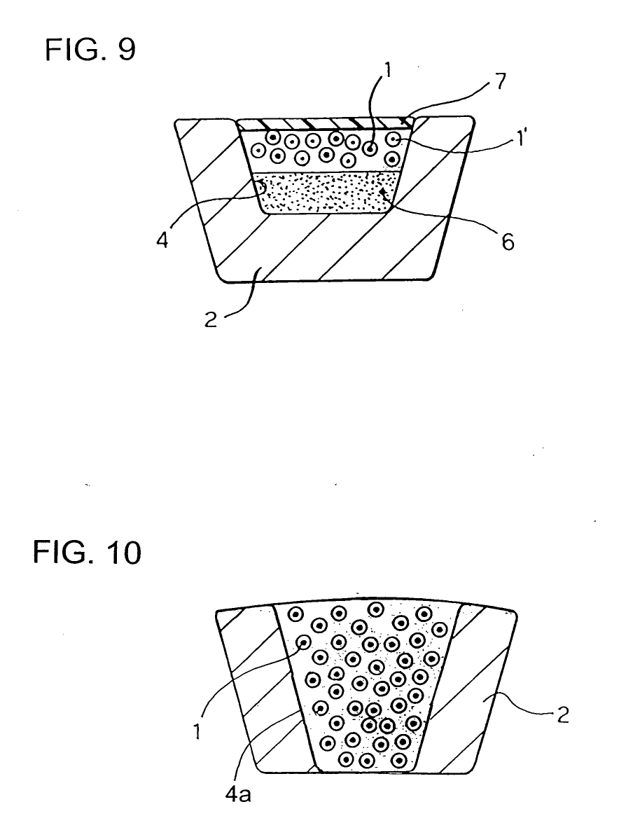

The flexibility of the corresponding mechanism is illustrated, purely by way of example, in FIGS. 3 to 10. In particular, FIG. 3 basically re-proposes, in a schematic way, the solution of FIG. 2, with the nanoparticles 1 constituting a filling directly contained in the recess 4 of the carrier. FIG. 4 relates, instead, to a solution in which in the recess 4 there are present two different species or kinds of nanoparticles, one of which is designated by 1 and the other by 1′.

The two species are differentiated in at least one of the characteristics typical of the core 1a and/or of the envelope 1b, such as, for example, at least one of the following characteristics:
bio-erodible nature of the envelope 1b;
time of erosion of the envelope 1b;
permeability of the envelope 1b to the active principle contained in the respective core 1a;
thickness of the envelope 1b;
stratified structure of the envelope 1b; and
characteristics of selective affinity attraction of the material constituting the envelope 1b in regards to said at least one first region and one second region.

The two species of nanoparticles 1 and 1′ are mixed together and again constitute a free filling of the recess 4.

In the example of FIG. 5, two types of nanoparticles 1, 1′ are present. However, instead of being mixed together as shown in FIG. 4, in FIG. 5 the nanoparticles form two layers, an outer layer comprising nanoparticles 1′ and an inner layer comprising nanoparticles 1. The solution of FIG. 5 preferably is used in applications in which the active principle conveyed by nanoparticles 1′ are desired to be delivered before to the active principle conveyed by the nanoparticles 1.

The solutions illustrated in FIGS. 6 to 8 essentially correspond to the same solutions as those illustrated in FIGS. 3 to 5, respectively, with the difference that, in the case of the solutions of FIGS. 6 to 8, the nanoparticles 1, 1′ do not simply constitute a free filling of the respective recess but are instead received in one or more corresponding polymeric matrices 5, 5′.

FIG. 6 illustrates one type of particle 1 within polymeric matrix 5 in recess 4. FIG. 7 illustrates particles 1 and 1′ within polymeric matrix 5. FIG. 8 shows particles 1 within polymeric matrix 5 in a layer beneath a layer of particles 1′ in matrix 5′. The layered arrangement is similar to that described for FIG. 5.

FIG. 9 illustrates yet another possible embodiment of the invention. In this solution, inside the recess 4 there are arranged, starting from the bottom of the recess 4, the following:
a layer of active principle (for example, a drug 6) set in a respective polymeric matrix;
a layer comprising two species of nanoparticles 1, 1′ which are mixed together (and are possibly incorporated in a respective polymeric matrix); and
a top or cover layer 7 of bio-erodible polymeric material which closes, in the manner of an operculum, the top aperture of the recess 4.

The presence of the cover layer of polymeric material 7 is designed to cause delivery of the active principles in the underlying recess 4 to start only after the cover layer 7 has been eroded and/or rendered permeable in regard to said active principles.

FIG. 10 illustrates a recess or reservoir 4a that extends through the thickness of element 2 of the stent having nanoparticles 1 within recess 4a. It is to be understood that the nanoparticles could be mixed with other species or kinds of nanoparticles, stratified, be placed in a polymeric matrix, and/or be covered with a cover layer, as described for the embodiments above.

The stent acting as a carrier body can therefore comprise a plurality of recesses or reservoirs 4 that have the function of reservoirs, the plurality comprising at least one first recess 4 and at least one second recess 4 which have associated thereto respective masses of polymeric material which are differentiated from one another in at least one characteristic chosen in the group of:
function of the polymeric mass as a matrix or as a closing cover layer of the reservoir;
bio-erodibility of the polymeric mass;
time of erosion of the polymeric mass;
permeability of the polymeric mass to the active principle or principles conveyed by the nanoparticles;
thickness of the polymeric mass; and
stratified structure of the polymeric mass.
   As regards the characteristics of the recesses or reservoirs 4, the delivery mechanism described can draw considerable advantage in terms of flexibility from the possibility of intervening selectively on parameters such as:
size and shape of the individual recess;
location of the recess on the carrier body;
blind (i.e., opening to one surface) or through (i.e., opening to both inner and outer surfaces) character of the recess.

In a particularly preferred way, the carrier has surfaces, an outer one and an inner one, with respect to the site of intraluminal implantation, and the recesses are located on the outer surface.

Of course, without prejudice to the principle of the invention, the details of construction and the embodiments may vary widely with respect to what is described and illustrated herein, without thereby departing from the scope of the present invention as defined in the claims which follow.

1. A carrier for delivering at least one active principle at an intraluminal site, the intraluminal site having at least a first region and a second region, the carrier comprising:
a carrier body sized to be conveyed to the intraluminal site, the carrier body having an inner surface and an outer surface and at least one reservoir formed into the outer surface; and
a plurality of nanoparticles contained within the at least one reservoir, each nanoparticle including an outer envelope and containing the active principle, the outer envelope comprising at least a first substance having characteristics of affinity of preferential attraction to the second region as compared to the first region.

2. The carrier of claim 1 wherein each of the plurality of nanoparticles comprises a core.

3. The carrier of claim 3 wherein the core comprises the active principle.

4. The carrier of claim 3 wherein the outer envelope is permeable to the active principle in the core.

5. The carrier of claim 1 wherein the outer envelope comprises bio-erodible material.

6. The carrier of claim 1 wherein the outer envelope comprises a stratified structure.

7. The carrier of claim 1 wherein the at least one reservoir comprises a plurality of reservoirs.

8. The carrier of claim 7 wherein the plurality of reservoirs contains at least two different species of nanoparticles, the two species being differentiated from one another by at least one characteristic of the active principle or of the first substance.

9. The carrier of claim 1 wherein the active principle is selected from one of anti-inflammatory agents, antineoplastic agents, vessel-wall repair agents, and restenosis-antagonist agents.

10. The carrier of claim 1 wherein the first substance is selected from one of functional groups of recognition of muscle cells, peptide sequences of recognition, proteins of recognition, antibodies, and antibody fragments.

11. The carrier of claim 1 wherein the at least first substance comprises a sequence of the arginine-glycine-aspartic acid (RGD) type.

12. The carrier of claim 1 wherein the active principle comprises a material that promotes re-growth of the intima of the endothelium.

13. The carrier of claim 1 wherein the at least first substance comprises a lipid.

14. The carrier of claim 1 wherein the at least first substance comprises stearic acid.

15. The carrier of claim 1 further comprising a polymeric material acting as a dispersion matrix within the at least one reservoir.

16. The carrier of claim 15 wherein the polymeric material is bio-erodible.

17. The carrier of claim 1 further comprising a polymeric material acting as a cover layer over the at least one reservoir.

18. The carrier of claim 17 wherein the polymeric material is permeable to the active principle.

19. The carrier of claim 1 further comprising inner and outer surfaces, wherein the at least one reservoir is located on the outer surface of the carrier.

20. A stent for delivering at least one active principle at an intraluminal site, the intraluminal site having at least a first region and a second region, the stent comprising:
a body configured to be expandable from a delivery configuration to a deployed configuration, the body being sized to be delivered to the intraluminal site in the delivery configuration, the body having an interior surface and an exterior surface and having at least one reservoir defined by the exterior surface; and
a plurality of nanoparticles contained within the at least one reservoir, each nanoparticle including an outer envelope and containing the active principle, the outer envelope comprising at least a first substance having characteristics of affinity of preferential attraction to the second region as compared to the first region.

21. The stent of claim 20 wherein each of the plurality of nanoparticles comprises a core.

22. The stent of claim 21 wherein the core comprises the active principle.

23. The stent of claim 22 wherein the outer envelope is permeable to the active principle in the core.

24. The stent of claim 20 wherein the outer envelope comprises bio-erodible material.

25. The stent of claim 20 wherein the outer envelope comprises a stratified structure.

26. The stent of claim 20 wherein the at least one reservoir comprises a plurality of reservoirs.

27. The stent of claim 26 wherein the plurality of reservoirs contains at least two different species of nanoparticles, the two species being differentiated from one another by one of at least one characteristic of the active principle or of the first substance.

28. The stent of claim 20 wherein the active principle is selected from one of anti-inflammatory agents, antineoplastic agents, vessel-wall repair agents, and restenosis-antagonist agents.

29. The stent of claim 20 wherein the first substance is selected from one of functional groups of recognition of muscle cells, peptide sequences of recognition, proteins of recognition, antibodies, and antibody fragments.

30. The stent of claim 20 wherein the at least first substance comprises a sequence of the arginine-glycine-aspartic acid (RGD) type.

31. The stent of claim 20 wherein the active principle comprises a material that promotes re-growth of the intima of the endothelium.

32. The stent of claim 20 wherein the at least first substance comprises a lipid.

33. The stent of claim 20 wherein the at least first substance comprises stearic acid.

34. The stent of claim 20 further comprising a polymeric material acting as a dispersion matrix within the at least one reservoir.

35. The stent of claim 34 wherein the polymeric material is bio-erodible.

36. The stent of claim 20 further comprising a polymeric material acting as a cover layer over the at least one reservoir.

37. The stent of claim 36 wherein the polymeric material is permeable to the active principle.

38-56. (canceled)

57. A kit for delivering at least one active principle at a treatment site within the lumen of a vessel, the site having at least a first region and a second region, the kit comprising:
a carrier body sized to be conveyed through the lumen of the vessel to the treatment site, the carrier body having an inner surface and an outer surface, the outer surface being shaped to define at least one reservoir;
a plurality of nanoparticles contained within the at least one reservoir, each nanoparticle including an outer envelope and containing the active principle, the outer envelope comprising at least a first substance having characteristics of affinity of preferential attraction to the second region as compared to the first region; and
a delivery device for advancing the carrier body through the lumen to the treatment site.

58. The kit of claim 57 wherein the delivery device comprises a catheter.

59. The kit of claim 58 wherein the catheter comprises a balloon catheter.

60. The kit of claim 57 wherein the carrier body comprises a stent.

61. The kit of claim 57 wherein the nanoparticle further comprises a core.

62. The kit of claim 61 wherein the core comprises active principle.

63. The kit of claim 62 wherein the outer envelope is permeable to the active principle in the core.

64. The kit of claim 57 wherein the outer envelope comprises bio-erodible material.

65. The kit of claim 57 wherein the outer envelope comprises a stratified structure.

66. The kit of claim 57 wherein the at least one reservoir comprises a plurality of reservoirs.

67. The kit of claim 66 wherein the plurality of reservoirs contains at least two different species of nanoparticles, the two species being differentiated from one another by at least one characteristic of the active principle or of the first substance.

68. The kit of claim 57 wherein the active principle is selected from one of anti-inflammatory agents, antineoplastic agents, vessel-wall repair agents, and restenosis-antagonist agents.

69. The kit of claim 57 wherein the first substance is selected from one of functional groups of recognition of muscle cells, peptide sequences of recognition, proteins of recognition, antibodies, and antibody fragments.

70. The kit of claim 57 wherein the at least first substance comprises a sequence of the arginine-glycine-aspartic acid (RGD) type.

71. The kit of claim 57 wherein the active principle comprises a material that promotes re-growth of the intima of the endothelium.

72. The kit of claim 57 wherein the at least first substance comprises a lipid.

73-80. (canceled)

81. The kit of claim 57 wherein the at least first substance comprises stearic acid.

82. The kit of claim 57 further comprising a polymeric material acting as a dispersion matrix within the at least one reservoir.

83. The kit of claim 82 wherein the polymeric material is bio-erodible.

84. The kit of claim 57 further comprising a polymeric material acting as a cover layer over the at least one reservoir.

85. The kit of claim 84 wherein the polymeric material is permeable to the active principle.

86. A carrier for delivering at least one active principle at an intraluminal site, the intraluminal site having at least a first region and a second region, the carrier comprising:
a carrier body sized to be conveyed to the intraluminal site, the carrier body having an inner surface and an outer surface and a plurality reservoirs formed into the outer surface; and
at least two different species of nanoparticles contained within the plurality of reservoirs, each nanoparticle including an outer envelope and containing the active principle, the outer envelope comprising at least a first substance having characteristics of affinity of preferential attraction to the second region as compared to the first region, and the at least two different species of nanoparticles being differentiated from one another by at least one characteristic of the active principle or of the first substance.

87. A stent for delivering at least one active principle at an intraluminal site, the intraluminal site having at least a first region and a second region, the stent comprising:
a body configured to be expandable from a delivery configuration to a deployed configuration, the body being sized to be delivered to the intraluminal site in the delivery configuration, the body having an interior surface and an exterior surface and having a plurality of reservoirs defined by the exterior surface; and
at least two different species of nanoparticles contained within the plurality of reservoirs, each nanoparticle including an outer envelope and containing the active principle, the outer envelope comprising at least a first substance having characteristics of affinity of preferential attraction to the second region as compared to the first region, and the at least two different species of nanoparticles being differentiated from one another by at least one characteristic of the active principle or of the first substance.

88. A kit for delivering at least one active principle at a treatment site within the lumen of a vessel, the site having at least a first region and a second region, the kit comprising:
a carrier body sized to be conveyed through the lumen of the vessel to the treatment site, the carrier body having a plurality of reservoirs;
at least two different species of nanoparticles contained within the plurality of reservoirs, each nanoparticle including an outer envelope and containing the active principle, the outer envelope comprising at least a first substance having characteristics of affinity of preferential attraction to the second region as compared to the first region, and the two different species being differentiated from one another by at least one characteristic of the active principle or of the first substance; and
a delivery device for advancing the carrier body through the lumen to the treatment site.
